(19) United States
(12) Patent Application Publication
(10) Pub. No.: US 2007/0135830 A1
(43) Pub. Date: Jun. 14, 2007

(54) Flexible tip

(75) Inventor: Darin G. Schaeffer, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 10/591,367

(22) PCT Filed: Oct. 5, 2005
(86) PCT No.: PCT/US05/36245

§ 371 Date: Aug. 31, 2006

Related U.S. Application Data
(60) Provisional application No. 60/616,416, filed on Oct. 6, 2004.

(51) Int. Cl. A61M 29/00

(52) U.S. Cl. 606/192; 604/96.01; 604/523

(57) ABSTRACT

An elongate flexible catheter tip is disclosed herein. This catheter tip may include a longitudinal axis extending between a proximal end and a distal end. It may further comprise a corrugated region located between the proximal end and the distal end. The elongate flexible catheter tip, as disclosed herein, may be employed with a large variety of dilation catheters.

### BACKGROUND

Catheters are tube-like medical devices that may be inserted into a body cavity, organ, or blood vessel for diagnostic or therapeutic reasons. Catheters may be designed for insertion into the vasculature and are available for a wide variety of purposes, including diagnosis, interventional therapy, drug delivery, drainage, perfusion, and the like. They may also be useful for other procedures, such as gynecological procedures, cardiac procedures, general interventional radiology procedures, and the like. Catheters for each of these and other purposes can be introduced to numerous target sites within a patient's body by guiding the catheter through an incision made in the patient's skin.

Catheters generally have an elongated, flexible catheter body enclosing one or more lumens. The lumen(s) may extend from the proximal end to the distal end of the catheter body. The diameter of the lumen(s) may vary throughout the length of the catheter, such as when the lumens have a larger diameter at the proximal end than at the distal end, or the diameter of the lumen(s) may be uniform. When an inner body is placed substantially in the center of the outermost body, the lumens may be coaxially arranged. The catheter body may be relatively straight, inherently curved, or curved by insertion of a curved stiffening wire or wire guide through a catheter lumen. The catheter body may assume a straight or linear configuration, when free from external bending forces. The catheter body may be highly flexible and thus capable of passing through the tortuous twists and turns of a patient's vasculature. In some cases, the catheter body may have a shaped distal end portion including curves and bends, which are selected to facilitate introduction and placement of the catheter in the vascular system. A particular geometry of curves and/or bends may be selected to accommodate the intended use of the catheter. The distal end of the catheter may also be equipped with an inflatable balloon to expand a medical device, such as a stent, and/or to dilate a vessel.

A lumen, extending through the length of a catheter, is often designed to enable the catheter to be employed in conjunction with a wire guide. This type of catheter is generally referred to as an over-the-wire catheter. There are many different types of over-the-wire catheters, including those adapted for dilation and stent delivery.

The wire guide is a small wire that is inserted into the patient in advance of an over-the-wire catheter. This wire may be inserted through a patient's skin and then fed along the desired conduit, such as a blood vessel, until it reaches the desired location. The smaller diameter and the malleability of the wire guide generally make it easier to feed through a potentially tortuous conduit, compared to the catheter itself. Once the wire guide has reached the location where treatment is to occur, the distal end of the over-the-wire catheter is fed over the proximal end of the wire guide. Next, the catheter may be advanced over the wire guide, and to the desired location, by applying force to the proximal end of the catheter.

The distal end of the catheter body may terminate in a catheter tip. There are a wide variety of different catheter tips, including rotating tips, shaped tips, cutting tips, and soft tips. Many of these distal catheter tips are designed to reduce the potential for trauma, such as the abrasion or puncture of the conduit, such as a blood vessel.

Reducing the potential for trauma to a conduit can involve modifying a variety of catheter tip design features. For example, some catheter tips are rounded and/or soft, which may reduce the risk of abrasion by allowing the catheter to more easily move through the conduit. Other catheter tips are designed to be more flexible and/or compressible. A flexible tip may be more likely to deflect if forced against a wall of the conduit, rather than puncturing the conduit wall. Similarly a compressible tip may be less likely to puncture the wall of the conduit, since such a tip may reduce the force with which a catheter contacts an obstruction or the conduit wall. Additionally, a flexible and/or compressible catheter tip may allow the catheter to be more easily advanced over a wire guide. This is particularly important when the catheter is being advanced over a wire guide containing one or more sharp curves or turns. If the catheter can be easily advanced over a wire guide it may decrease the risk of abrasion and/or puncture.

### BRIEF SUMMARY

In one aspect of the invention, there is an elongate flexible catheter tip comprising a longitudinal axis extending between a proximal end and a distal end. These aspects may further include a corrugated region located between the proximal end and the distal end.

In a further aspect of the invention, there is an elongate flexible tip comprising a proximal tip end, a distal tip end, and a corrugated region located between the proximal tip end and the distal tip end, wherein the proximal tip end is adjacently attached to an inner distal end of a dilation catheter. The dilation catheter comprises an elongate outer body comprising a longitudinal axis extending between an outer proximal end and an outer distal end. The dilation catheter further comprises an elongate inner body having a proximal region, located within the outer body and extending between the outer proximal end and the outer distal end, a distal region extending past the outer distal end and comprising the inner distal end, and an inner lumen contained within the inner body. These aspects may further include an outer lumen defined by the outer body and the inner body, These aspects may also include a balloon comprising a proximal balloon leg attached to the outer distal end, a distal balloon leg attached to a distal end of the dilation catheter, and a balloon cavity defined by the proximal balloon leg and the distal balloon leg and in fluid communication with the outer lumen.

In a further aspect of the invention, there is an elongate flexible tip comprising a longitudinal axis extending between a proximal tip end and a distal tip end. In addition, a corrugated region is located between the proximal tip end and the distal tip end. These aspects may also include a tip lumen defined by the elongate flexible tip, such that the tip lumen is aligned with a wire guide lumen. Furthermore, the proximal tip end is adjacently attached to a distal body end of a dilation catheter. The dilation catheter comprises an elongate body with a longitudinal axis extending between a proximal body end and the distal body end, an inflation lumen and the wire guide lumen, wherein the inflation lumen and the wire guide lumen are parallel and are defined by the elongate body, and an intermediate region positioned between the proximal body end and the distal body end. These aspects may further include a balloon comprising a proximal balloon leg attached to the intermediate region, a distal balloon leg adjacently attached to the distal body end, and a balloon cavity defined by the proximal balloon leg and the distal balloon leg, and in fluid communication with the inflation lumen.

### BRIEF DESCRIPTION OF THE DRAWINGS

The invention can be better understood with reference to the following drawings and description. The components in the figures are not necessarily to scale, emphasis instead being placed upon illustrating the principles of the invention.

FIG. 1A illustrates, by means of a longitudinal cross-sectional view, an example of an elongate flexible catheter tip with a helical corrugation.

FIG. 1B illustrates, by means of a longitudinal three dimensional view, an example of an elongate flexible tip with a helical corrugation.

FIG. 1C illustrates, by means of a longitudinal cross-sectional view, an example of an elongate flexible catheter tip with an accordion corrugation.

FIG. 1D illustrates, by means of a longitudinal cross-sectional view, an example of an elongate flexible catheter tip with an accordion corrugation.

FIG. 2 illustrates, by means of a longitudinal cross-sectional view, an example of an elongate flexible catheter tip, wherein a distal end of the flexible tip includes a rounded end.

FIG. 3 illustrates, by means of a longitudinal cross-sectional view, an example of an elongate dilation catheter with an elongate flexible tip, wherein a proximal tip end is adjacently attached to a distal balloon leg and an inner distal end.

FIG. 4 illustrates, by means of a longitudinal cross-sectional view, an example of an elongate dilation catheter, wherein the catheter includes an elongate flexible tip.

FIG. 5 illustrates, by means of a longitudinal cross-sectional view, an example of an elongate dilation catheter with an elongate flexible tip, wherein the flexible tip is attached to an inner distal end, forming a tip-end attachment.

FIG. 6 illustrates, by means of a longitudinal cross-sectional view, an example of an elongate dilation catheter with an elongate flexible tip, wherein an elongate inner body is reinforced with a braided coil.

FIG. 7 illustrates, by means of a longitudinal cross-sectional view, an elongate dilation catheter with a flexible tip, wherein the flexible tip is attached to a distal face.

FIG. 8 illustrates, by means of a longitudinal cross-sectional view, an example of an elongate dilation catheter with an elongate flexible tip, wherein an elongate inner body is composed of an inner material and an outer material.

FIG. 9 illustrates, by means of a longitudinal cross-sectional view, an example of an elongate dilation catheter with an elongate flexible tip, wherein the flexible tip is attached via an external mounting shoulder.

FIG. 10 illustrates, by means of a longitudinal cross-sectional view, an example of an elongate dilation catheter with an elongate flexible tip, wherein a proximal tip end is adjacently attached to a distal balloon leg and a distal body end.

FIG. 11 illustrates, by means of a longitudinal cross-sectional view, an example of an elongate dilation catheter, wherein the catheter may include an elongate flexible tip.

FIG. 12 illustrates, by means of a longitudinal cross-sectional view, an example of an elongate dilation catheter with an elongate flexible tip, wherein a proximal tip end is adjacently attached to a distal body end, forming a tip-end attachment.

FIG. 13 illustrates, by means of a longitudinal cross-sectional view, an example of an elongate dilation catheter with an elongate flexible tip, wherein a proximal tip end may be adjacently attached to a distal body end via a distal face.

FIG. 14 illustrates, by means of a longitudinal cross-sectional view, an example of an elongate dilation catheter with an elongate flexible tip, wherein a proximal tip end may be adjacently attached to a distal body end via an external mounting shoulder.

FIG. 15 illustrates a cross-sectional view through lines A-A of FIGS. 3 to 9.

FIG. 16 illustrates a cross-sectional view through lines B-B of FIGS. 10 to 14.

### DETAILED DESCRIPTION

Reducing the potential for trauma to a conduit can involve modifying a variety of catheter tip design features. For example, some catheter tips are rounded and/or soft, while others are designed to be more flexible and/or compressible. A flexible tip may be more likely to deflect if forced against a wall of the conduit, while a compressible tip may be less likely to puncture the wall of the conduit. Additionally, a flexible and/or compressible catheter tip may allow the catheter to be more easily advanced over a wire guide. This is particularly important when the catheter is being advanced over a wire guide containing one or more sharp curves or turns. If the catheter can be easily advanced over a wire guide it may further decrease the risk of abrasion and/or puncture. The following examples illustrate an elongate flexible catheter tip and the incorporation of this tip in a variety of catheters.

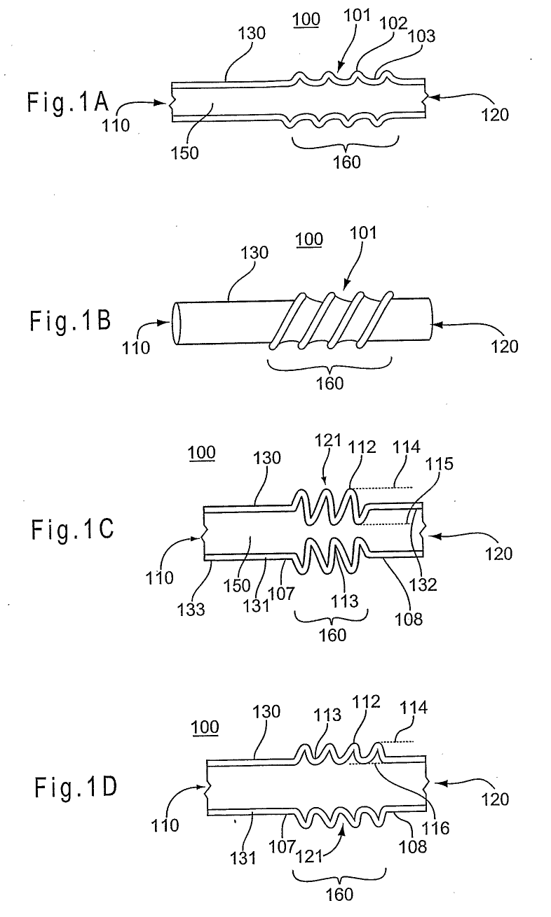

FIG. 1A illustrates, by means of a longitudinal cross-sectional view, an example of an elongate flexible tip 100 with a helical corrugation 101. The helical corrugation 101 may comprise a spiral ridge 102 and a corresponding spiral groove 103 that are both continuous along the length of a corrugated region 160.

The flexible tip 100 may have a longitudinal axis extending between a proximal end 110 and a distal end 120. The flexible tip 100 may be made from any suitable material, including, but not limited to, polyethylene, polyamides, polyethers, polyether-block co-polyamide polymers, polyvinyl chloride (PVC), polystyrene, silicon co-polymer, polyolefin, polyurethane and combinations thereof. For example, the flexible tip 100 may be made from a polyether-block co-polyamide polymers, which may include a copolymer of amide monomers copolymerized with polyether monomers. Because the amide monomers may have greater structural “rigidity” in comparison to the polyether monomers, the rigidity of the resulting flexible tip 100 to deformation, such as bending or stretching, may be altered. The flexible tip 100 may also be made from laminates of these materials. One example of a suitable polyether-block co-polyamide polymer from which the flexible tip 100 can be made is PEBAX®, which is available from Elf Atofina, Philadelphia, Pa. In one aspect, a blend of PEBAX® polyether-block co-polyamide polymers may be used. In another configuration, the flexible tip 100 may be made from a polyamide polymer, such as nylon 12.

In one configuration, the flexible tip 100 may comprise a tube member 130. The tube member 130 may define a lumen 150, extending longitudinally from the proximal end 110 to the distal end 120 of the flexible tip 100. In another configuration, the flexible tip 100 may be solid, such that no lumen is present (not shown). The flexible tip 100 and the corrugated region 160 may both vary in length. For example, the flexible tip 100 may range from about 2 mm to about 2 cm in length. In a preferred configuration, the flexible tip 100 may range from about 3 mm to about 1 cm in length.

The flexible tip 100 may include the corrugated region 160, located between the proximal end 110 and the distal end 120. The corrugated region 160 may be produced by applying a force to the precursor of tube member 130. For example, the precursor to tube member 130 may be placed over a mandrel and the force may be applied in-line with the precursor of tube member 130. The application of this type of force may cause the tube member 130 to buckle, producing the corrugated region 160. In one configuration, the precursor of tube member 130 may be heated, using steam for example, during or after application of the force. Application of the heat during this process may provide the flexible tip 100 with memory, such that the flexible tip 100 may maintain the corrugated shape of the corrugated region 160 more efficiently.

The corrugated region 160 may possess several inherent features, including flexibility, deflectability, compressibility and conformability, which may contribute to the atraumatic nature of the flexible tip. For example, when a wire guide is not present, the corrugated region 160 may impart flexibility to the tip 100. This flexibility may allow the tip 100 to preferentially deflect, if forced against an obstruction, such as a blood vessel wall, rather than puncturing the obstruction.

The corrugated region 160 may also contribute to the compressibility of the flexible tip 100. When the flexible tip 100 contacts an obstruction, the compressibility of the flexible tip 100 may reduce the amount of force transferred to the obstruction by the flexible tip 100. The corrugated region 160 of the flexible tip 100 may also provide a catheter tip which will better conform to the wire guide. This may be especially true if the catheter is being advanced over the wire guide containing sharp curves or bends. This conformability may result from the ability of the flexible tip to transfer torque around a sharp bend, when following the wire guide. The conformability, or the ability to transfer torque around a sharp bend, may allow a physician to more easily advance the corresponding catheter over the wire guide by reducing the amount of force that the physician must apply.

FIG. 1B illustrates, by means of a longitudinal three dimensional view, an example of an elongate flexible tip 100 with a helical corrugation 101.

FIG. 1C illustrates, by means of a longitudinal cross-sectional view, an example of an elongate flexible catheter tip 100 with an accordion corrugation 121. In this configuration, the corrugated region 160 may be defined by an adjacent proximal tube portion 107 and an adjacent distal tube portion 108 and may further comprise a plurality of ridges 112 that may be interspersed with a plurality of grooves 113. The elongate flexible catheter tip may also include ridges alone or grooves alone. The ridges 112 may have an outer diameter 114 that is greater than the outer diameter of the tube member 130 and/or the adjacent tube portions 107 and 108. The grooves may have an inner diameter 115 that is smaller than the inner diameter of the tube member 130 and/or the adjacent tube portions 107 and 108.

The tube member 130 and the corrugated region 160 may also comprise a tube wall 131 that is defined by an inner lumen surface 132 and an outer tube surface 133. In this configuration, the tube wall 131 may have a substantially uniform thickness throughout the length of the tube member 130. That is, the thickness of the tube wall 131 may be substantially the same, whether the thickness is measured along the adjacent portions 107, 108 or at the ridges 114 or grooves 113 of the corrugated region 160.

FIG. 1D illustrates, by means of a longitudinal cross-sectional view, an example of an elongate flexible catheter tip 100 with an accordion corrugation 121. In this configuration, the grooves 113 may have an inner diameter 116 that is substantially the same as the inner diameter of the tube member 130 and/or the adjacent tube portions 107 and 108.

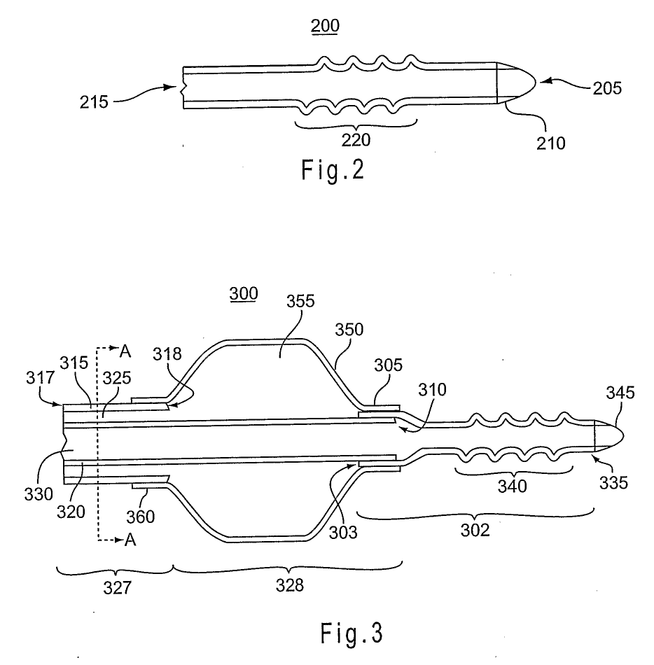

FIG. 2 illustrates, by means of a longitudinal cross-sectional view, an example of an elongate flexible tip 200, wherein a distal end 205 of the flexible tip 200 includes a rounded end 210. The flexible tip 200 may have a longitudinal axis extending between a proximal end 215 and the distal end 205. The flexible tip 200 may include a corrugated region 220, which may be located between the proximal end 215 and the distal end 205.

The rounded end 210 may enhance the deflectability of the flexible tip 200. In one configuration, the rounded end 210 may be integral with or formed from the flexible tip 200. In another configuration, the rounded end 210 may be a separate piece or component that is attached to the distal end of the flexible tip 200. Thus, the rounded end 210 may be made of a material which is of a lower durometer than the flexible tip 200, thus providing enhanced softness for the rounded end 210. In another embodiment, the rounded end 210 may be a separate piece of component that possesses an inner diameter that is smaller than an inner diameter of the flexible tip 200. The enhanced softness of the rounded end 210 may help to prevent abrasions or snagging on obstructions, as the flexible tip 200 is advanced through a conduit. In addition, it may further improve the deflectability of the flexible tip 200.

A catheter having a flexible tip containing a corrugated region may be employed for a variety of applications, including diagnosis, interventional therapy, drug delivery, drainage, perfusion, and the like. Such a catheter may also be useful for other procedures, such as gynecological procedures, cardiac procedures, general interventional radiology procedures, and the like. Furthermore a flexible tip containing a corrugated region may be incorporated into a dilation catheter, wherein a balloon may be attached to the catheter (and/or tip) using a variety of attachment configurations.

For example, FIG. 3 illustrates, by means of a longitudinal cross-sectional view, an example of an elongate dilation catheter 300 with an elongate flexible tip 302, wherein a proximal tip end 303 is adjacently attached to a distal balloon leg 305 and an inner distal end 310. The dilation catheter 300 may be composed of an elongate outer body 315 and an elongate inner body 320. The outer body 315 may have a longitudinal axis extending between an outer proximal end 317 and an outer distal end 318. The outer body 315 and the inner body 320 may define an outer lumen 325 therebetween. The inner body 320 may include a proximal region 327, located within the outer body 315 and extending between the outer proximal end 317 and the outer distal end 318. Furthermore, the inner body 320 may have a distal region 328, extending past the outer distal end 318 and comprising the inner distal end 310. The inner body 320 may contain a single inner lumen 330.

The elongate flexible tip 302 may include the proximal tip end 303, a distal tip end 335, and a corrugated region 340 located between the proximal tip end 303 and the distal tip end 335. The distal tip end 335 may be integral with a rounded distal end 345 or the rounded distal end 345 may be a separate piece or component that is attached to the distal tip end 335.

The dilation catheter 300 may be fitted with a balloon 350. The balloon 350 may have a balloon cavity 355 in fluid communication with the outer lumen 325, wherein the balloon cavity 355 may be defined by a proximal balloon leg 360 and the distal balloon leg 305. The proximal balloon leg 360 may be attached to the outer distal end 318. The proximal tip end 303, the distal balloon leg 305, and the inner distal end 310 may be adjacently attached. In one configuration, the proximal tip end 303 may be sandwiched between the distal balloon leg 305 and the inner distal end 310.

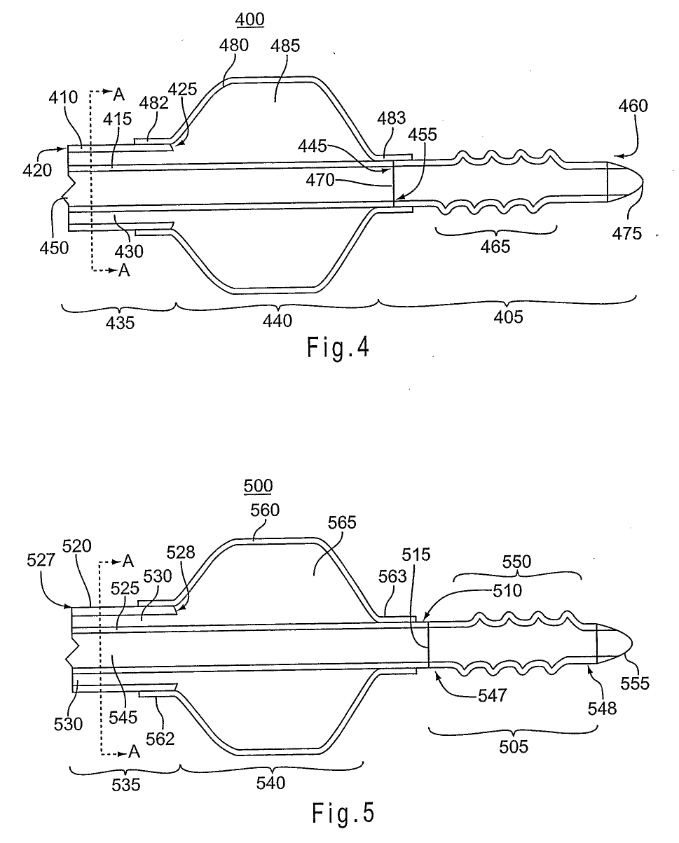

In a further example, FIG. 4 illustrates, by means of a longitudinal cross-sectional view, an example of an elongate dilation catheter 400, wherein the catheter 400 may include an elongate flexible tip 405. The dilation catheter 400 may have an elongate outer body 410 and an elongate inner body 415. The outer body 410 may have a longitudinal axis extending between an outer proximal end 420 and an outer distal end 425. The outer body 410 and the inner body 415 may define an outer lumen 430 therebetween. The inner body 415 may include a proximal region 435, located within the outer body 410 and extending between the outer proximal end 420 and the outer distal end 425. Furthermore, the inner body 415 may have a distal region 440, extending past the outer distal end 425 and comprising an inner distal end 445. The inner body 415 may contain a single inner lumen 450.

The elongate flexible tip 405 may include a proximal tip end 455, a distal tip end 460, and a corrugated region 465 located between the proximal tip end 455 and the distal tip end 460. In one configuration, the inner distal end 445 may be attached to the proximal tip end 455, forming a tip-end attachment 470. Furthermore, the distal tip end 460 may be integral with a rounded distal end 475. In another configuration, the rounded distal end 475 may be a separate piece or component that is attached to the distal tip end 460.

The dilation catheter 400 may be fitted with a balloon 480, having a proximal balloon leg 482 and a distal balloon leg 483. The proximal balloon leg 482 may be attached to the outer distal end 425. The distal balloon leg 483 may be attached adjacent to the tip-end attachment 470. It is worth noting that an “adjacent attachment”, as used throughout this document, includes an operable connection or a functional connection, such that there may be intervening layers between the two pieces, components or members that are being attached. Furthermore, “adjacently attached” and “attached adjacent to”, have the same meaning as that defined for an adjacent attachment. The balloon 480 may have a balloon cavity 485 in fluid communication with the outer lumen 430, wherein the balloon cavity 485 is defined by the distal balloon leg 482 and the proximal balloon leg 483.

In another example, FIG. 5 illustrates, by means of a longitudinal cross-sectional view, an example of an elongate dilation catheter 500 with an elongate flexible tip 505, wherein the tip 505 is attached to an inner distal end 510, forming a tip-end attachment 515. The dilation catheter 500 may be composed of an elongate outer body 520 and an elongate inner body 525. The outer body 520 may have a longitudinal axis extending between an outer proximal end 527 and an outer distal end 528. The outer body 520 and the inner body 525 may define an outer lumen 530 therebetween. The inner body 525 may include a proximal region 535, located within the outer body 520 and extending between the outer proximal end 527 and the outer distal end 528. Furthermore, the inner body 525 may have a distal region 540, extending past the outer distal end 528 and comprising the inner distal end 510. The inner body 525 may contain a single inner lumen 545.

The elongate flexible tip 505 may include a proximal tip end 547, a distal tip end 548, and a corrugated region 550 located between the proximal tip end 547 and the distal tip end 548. In one configuration, the inner distal end 510 may be attached adjacent to the proximal tip end 547, forming the tip-end attachment 515. Furthermore, the distal tip end 548 may be integral with a rounded distal end 555. In another configuration, the rounded distal end 555 may be a separate piece or component that is attached to the distal tip end 548.

The dilation catheter 500 also may be fitted with a balloon 560, having a proximal balloon leg 562 and a distal balloon leg 563. The proximal balloon leg 562 may be attached to the outer distal end 528, while the distal balloon leg 563 may be attached to the distal region 540. The balloon 560 may have a balloon cavity 565 in fluid communication with the outer lumen 530, wherein the balloon cavity 565 may be defined by the distal balloon leg 563 and the proximal balloon leg 562.

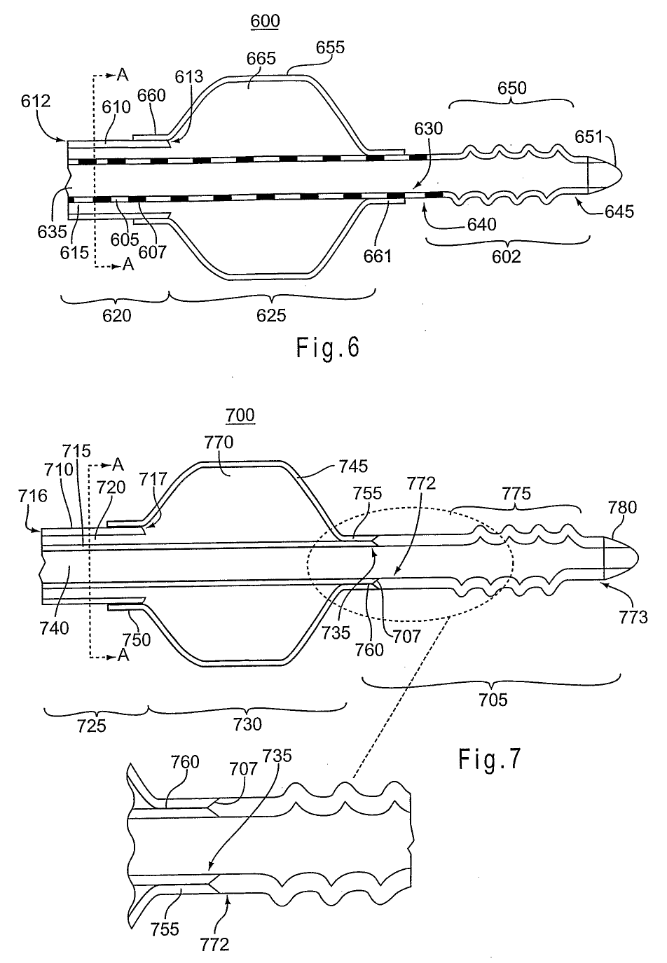

In an additional example, FIG. 6 illustrates, by means of a longitudinal cross-sectional view, an example of an elongate dilation catheter 600 with an elongate flexible tip 602, wherein an elongate inner body 605 is reinforced with a braided coil 607. The dilation catheter 600 may be composed of an elongate outer body 610 and the elongate inner body 605. The outer body 610 may have a longitudinal axis extending between an outer proximal end 612 and an outer distal end 613. The outer body 610 and the inner body 605 may define an outer lumen 615 therebetween. The inner body 605 may include a proximal region 620, located within the outer body 610 and extending between the outer proximal end 612 and the outer distal end 613. Furthermore, the inner body 605 may have a distal region 625, extending past the outer distal end 613 and comprising an inner distal end 630. The inner body 605 may contain a single inner lumen 635.

The braided coil 607 may serve to reinforce the inner body 605. The braided coil 607 may be imbedded in the inner body 605. In another configuration, the braided coil 607 may be adhered to the exterior or the interior of the inner body 605. The braided coil 607 may extend throughout the entirety of the inner body 605 or the braided coil 607 may only extend through a portion of the inner body 605. The tightness of the braided coil 607 may affect its flexibility and thus the flexibility of the inner body 605. If the tightness of the braided coil 607 is decreased, the flexibility of the braided coil 607, and the corresponding inner body 625, may be increased. The tightness of the braided coil 607 may be uniform throughout its length or it may vary throughout its length. The flexibility of the inner body 605 may vary throughout the length of the inner body 605. This may be accomplished by gradually decreasing the tightness of the braided coil 607 throughout the length of the inner body 605. In one configuration, the braided coil 607 may be constructed from a variety of materials. For example, materials may include stainless steel and nitinol.

The elongate flexible tip 602 may include a proximal tip end 640, a distal tip end 645, and a corrugated region 650 located between the proximal tip end 640 and the distal tip end 645. In one configuration, the proximal tip end 640 may be attached adjacent to the inner distal end 630 (not shown). In another configuration, the proximal tip end 640 may be integral with the inner distal end 630. Furthermore, the distal tip end 645 may be integral with a rounded distal end 651 or the rounded distal end 651 may be a separate piece or component that is attached to the distal tip end 645.

The dilation catheter 600 may be fitted with a balloon 655, having a proximal balloon leg 660 and a distal balloon leg 661. The proximal balloon leg 660 may be attached to the outer distal end 613. The distal balloon leg 661 may be attached to inner distal end 630. The balloon 655 may have a balloon cavity 665 in fluid communication with the outer lumen 615, wherein the balloon cavity 665 is defined by the proximal balloon leg 660 and the distal balloon leg 661.

In another example, FIG. 7 illustrates, by means of a longitudinal cross-sectional view, an example of an elongate dilation catheter 700 with a flexible tip 705, wherein the tip may be attached to a distal face 707. The dilation catheter 700 may be composed of an elongate outer body 710 and an elongate inner body 715. The outer body 710 may have a longitudinal axis extending between an outer proximal end 716 and an outer distal end 717. The outer body 710 and the inner body 715 may define an outer lumen 720 therebetween. The inner body 715 may include a proximal region 725, located within the outer body 710 and extending between the outer proximal end 716 and the outer distal end 717. Furthermore, the inner body 715 may have a distal region 730, extending past the outer distal end 717 and comprising an inner distal end 735. The inner body 715 may contain a single inner lumen 740.

The dilation catheter 700 also may be fitted with a balloon 745 having a proximal balloon leg 750 and a distal balloon leg 755. The proximal balloon leg 750 may be attached to the outer distal end 717. The distal balloon leg 755 may be attached to the inner distal end 735, forming a distal bonding region 760. The distal bonding region 760 may form the distal face 707. In one configuration, the distal face 707 may form an angular groove, circumscribing the inner lumen 740. In another configuration, the distal face 707 may be a flat surface, perpendicular to the longitudinal axis of the dilation catheter 700 (not shown). The balloon 745 may have a balloon cavity 770 in fluid communication with the outer lumen 720, wherein the balloon cavity 770 is defined by the distal balloon leg 755 and the proximal balloon leg 750.

The elongate flexible tip 705 may include a proximal tip end 772, a distal tip end 773, and a corrugated region 775 located between the proximal tip end 772 and the distal tip end 773. In one configuration, the proximal tip end 772 may be attached adjacent to distal face 707. Furthermore, the distal tip end 773 may be integral with a rounded distal end 780 or the rounded distal end 780 may be a separate piece or component that is attached to the distal tip end 773.

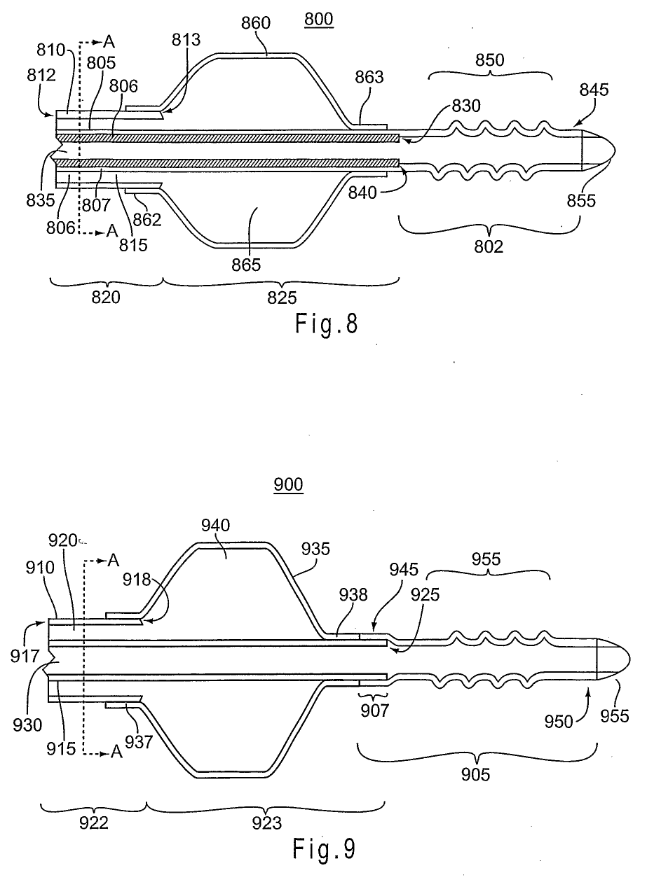

In an additional example, FIG. 8 illustrates, by means of a longitudinal cross-sectional view, an example of an elongate dilation catheter 800 with an elongate flexible tip 802, wherein an elongate inner body 805 is composed of an inner material 806 and an outer material 807. The dilation catheter 800 may be composed of an elongate outer body 810 and the elongate inner body 805. The outer body 810 may have a longitudinal axis extending between an outer proximal end 812 and an outer distal end 813. The outer body 810 and the inner body 805 may define an outer lumen 815 therebetween. The inner body 805 may include a proximal region 820, located within the outer body 810 and extending between the outer proximal end 812 and the outer distal end 813. Furthermore, the inner body 805 may have a distal region 825, extending past the outer distal end 813 and comprising an inner distal end 830. In addition, the outer material 807 may be of a lower durometer than the inner material 806. The inner body 805 may define a single inner lumen 835.

The elongate flexible tip 802 may include a proximal tip end 840, a distal tip end 845, and a corrugated region 850 located between the proximal tip end 840 and the distal tip end 845. The flexible tip 802 may be formed from the outer material 807 of the inner body 805. In one configuration, the distal tip end 845 may be integral with a rounded distal end 855 or the rounded distal end 855 may be a separate piece or component that is attached to the distal tip end 845.

The dilation catheter 800 may be fitted with a balloon 860, having a proximal balloon leg 862 and a distal balloon leg 863. The proximal balloon leg 862 may be attached to the outer distal end 813. The distal balloon leg 863 may be attached to the inner distal end 830. In another configuration, the distal balloon leg 863 may be attached to the flexible tip 802 (not shown). The balloon 860 may have a balloon cavity 865 in fluid communication with the outer lumen 815, wherein the balloon cavity 865 is defined by the proximal balloon leg 862 and the distal balloon leg 863.

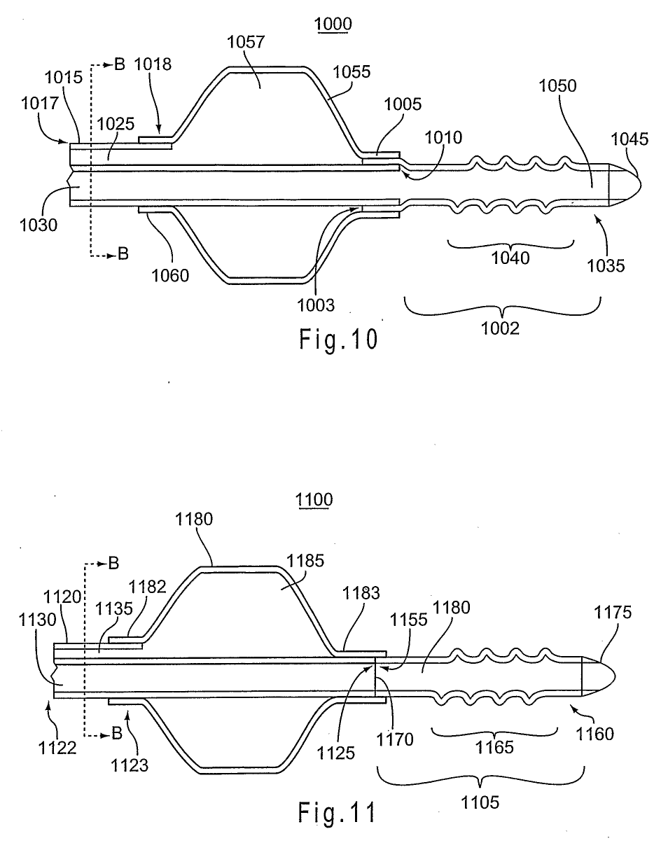

In another example, FIG. 9 illustrates, by means of a longitudinal cross-sectional view, an example of an elongate dilation catheter 900 with an elongate flexible tip 905, wherein the flexible tip 905 may be attached via an external mounting shoulder 907. The dilation catheter 900 may be composed of an elongate outer body 910 and an elongate inner body 915. The outer body 910 may have a longitudinal axis extending between an outer proximal end 917 and an outer distal end 918. The outer body 910 and the inner body 915 may define an outer lumen 920 therebetween. The inner body 915 may include a proximal region 922, located within the outer body 910 and extending between the outer proximal end 917 and the outer distal end 918. Furthermore, the inner body 915 may have a distal region 923, extending past the outer distal end 918 and comprising an inner distal end 925. The inner body 915 may contain a single inner lumen 930.

The dilation catheter 900 may be fitted with a balloon 935, having a proximal balloon leg 937 and a distal balloon leg 938. The proximal balloon leg 937 may be attached to the outer distal end 918. The distal balloon leg 938 may be attached to the distal region 923, forming the external mounting shoulder 907. The balloon 935 may have a balloon cavity 940 in fluid communication with the outer lumen 920, wherein the balloon cavity 940 is defined by the proximal balloon leg 937 and the distal balloon leg 938.

The elongate flexible tip 905 may include a proximal tip end 945, a distal tip end 950, and a corrugated region 955 located between the proximal tip end 945 and the distal tip end 950. The proximal tip end 945 may be attached to the external mounting shoulder 907. Furthermore, the distal tip end 950 may be integral with a rounded distal end 956 or the rounded distal end 955 may be a separate piece or component that is attached to the distal tip end 950.

In a further example, FIG. 10 illustrates, by means of a longitudinal cross-sectional view, an example of an elongate dilation catheter 1000 with an elongate flexible tip 1002, wherein a proximal tip end 1003 is adjacently attached to a distal balloon leg 1005 and a distal body end 1010. The dilation catheter 1000 may be composed of an elongate body 1015. The elongate body 1015 may have a longitudinal axis extending between a proximal body end 1017 and a distal body end 1010. In addition, the elongate body 1015 may define parallel dual lumens, an inflation lumen 1025 and a wire guide lumen 1030, wherein the lumens 1025 and 1030 extend longitudinally through the elongate body 1015. The wire guide lumen 1030 extends through the distal body end 1010, whereas the inflation lumen 1025 extends to an intermediate region 1018. The intermediate region 1018 may be positioned between the proximal body end 1017 and the distal body end 1010.

The elongate flexible tip 1002 may comprise a longitudinal axis extending between the proximal tip end 1003 and a distal tip end 1035. A corrugated region 1040 may be located between the proximal tip end 1003 and the distal tip end 1035. The distal tip end 1035 may be integral with a rounded distal end 1045 or the rounded distal end 1045 may be a separate piece or component that is attached to the distal tip end 1035. Additionally, the elongate flexible tip 1002 may comprise a tip lumen 1050 extending from the proximal tip end 1003 through the rounded distal end 1045, wherein the tip lumen 1050 is aligned with the wire guide lumen 1030.

The dilation catheter 1000 may be fitted with a balloon 1055. The balloon 1055 may have a balloon cavity 1057 in fluid communication with the inflation lumen 1025, wherein the balloon cavity 1057 may be defined by a proximal balloon leg 1060 and the distal balloon leg 1005. The proximal balloon leg 1060 may be attached to the intermediate region 1018. The distal balloon leg 1005 may be adjacently attached to the distal body end 1010 and the proximal tip end 1003. In one configuration, the proximal tip end 1003 may be sandwiched between the distal balloon leg 1005 and the distal body end 1010.

In a further example, FIG. 11 illustrates, by means of a longitudinal cross-sectional view, an example of an elongate dilation catheter 1100, wherein the catheter 1100 may include an elongate flexible tip 1105. The dilation catheter 1100 may be composed of an elongate body 1120. The elongate body 1120 may have a longitudinal axis extending between a proximal body end 1122, and a distal body end 1125. In addition, the elongate body 1120 may define parallel dual lumens, a wire guide lumen 1130 and an inflation lumen 1135, wherein the lumens 1130 and 1135 extend longitudinally through the elongate body 1120. The wire guide lumen 1130 extends through the distal body end 1125, whereas the inflation lumen 1135 extends to an intermediate region 1123. The intermediate region 1123 may be positioned between the proximal body end 1122 and the distal body end 1125.

The elongate flexible tip 1105 may comprise a longitudinal axis extending between a proximal tip end 1155 and a distal tip end 1160. A corrugated region 1165 may be located between the proximal tip end 1155 and the distal tip end 1160. In one configuration, the proximal tip end 1155 is adjacently attached to the distal body end 1125, forming a tip-end attachment 1170. The distal tip end 1160 may be integral with a rounded distal end 1175 or may be a separate piece or component that is attached to the distal tip end 1160. Additionally, the elongate flexible tip 1105 may comprise a tip lumen 1180 extending from the proximal tip end 1155 through the rounded distal end 1175, wherein the tip lumen 1180 is aligned with the wire guide lumen 1130.

The dilation catheter 1100 may be fitted with a balloon 1180, having a proximal balloon leg 1182 and a distal balloon leg 1183. The proximal balloon leg 1182 may be attached to the intermediate region 1123. The distal balloon leg 1183 may be adjacently attached to the distal body end 1125, the proximal tip end 1155, and the tip-end attachment 1170. The balloon 1180 may have a balloon cavity 1185 in fluid communication with the inflation lumen 1135, wherein the balloon cavity 1185 is defined by the distal balloon leg 1182 and the proximal balloon leg 1183.

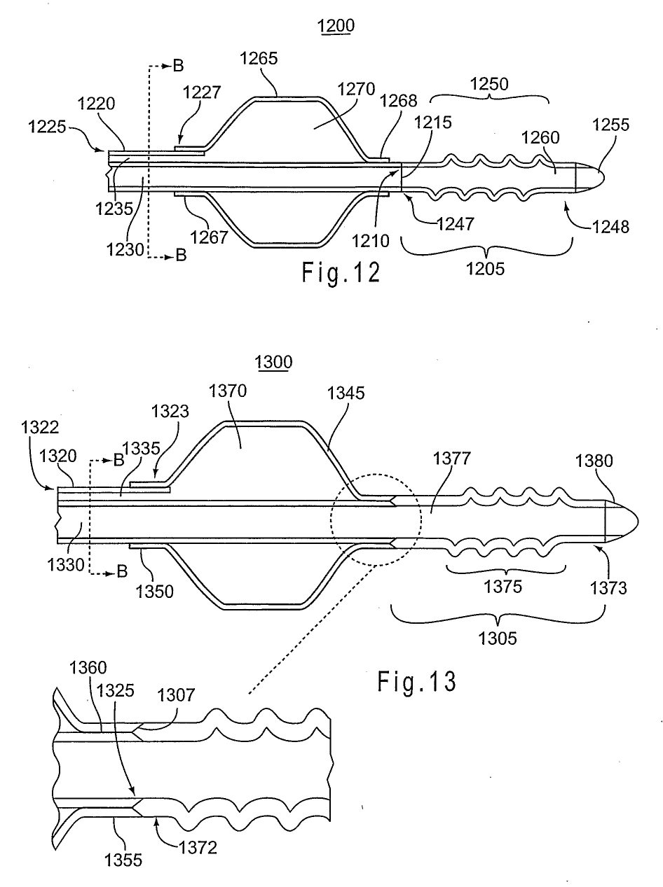

In another example, FIG. 12 illustrates, by means of a longitudinal cross-sectional view, an example of an elongate dilation catheter 1200 with an elongate flexible tip 1205, wherein a proximal tip end 1247 is adjacently attached to a distal body end 1210, forming a tip-end attachment 1215. The dilation catheter 1200 may be composed of an elongate body 1220. The elongate body 1220 may have a longitudinal axis extending between a proximal body end 1225, and the distal body end 1210. In addition, the elongate body 1210 may define parallel dual lumens, a wire guide lumen 1230 and an inflation lumen 1235, wherein the lumens 1230 and 1235 extend longitudinally through the elongate body 1220. The wire guide lumen 1230 extends through the distal body end 1210, whereas the inflation lumen 1235 extends to an intermediate region 1227. The intermediate region 1227 may be positioned between the proximal body end 1225 and the distal body end 1210.

The elongate flexible tip 1205 may comprise a longitudinal axis extending between the proximal tip end 1247 and a distal tip end 1248. A corrugated region 1250 may be located between the proximal tip end 1247 and the distal tip end 1248. The distal body end 1210 may be adjacently attached to the proximal tip end 1247, forming the tip-end attachment 1215. In one configuration, the distal tip end 1248 may be integral with a rounded distal end 1255 or the rounded distal end 1255 may be a separate piece or component that is attached to the distal tip end 1248. Additionally, the elongate flexible tip 1205 may comprise a tip lumen 1260 extending from the proximal tip end 1247 through the rounded distal end 1255, wherein the tip lumen 1260 is aligned with the wire guide lumen 1230.

The dilation catheter 1200 also may be fitted with a balloon 1265, having a proximal balloon leg 1267 and a distal balloon leg 1268. The proximal balloon leg 1267 may be attached to the intermediate region 1227, while the distal balloon leg 1268 may be adjacently attached to the distal body end 1210. The balloon 1265 may have a balloon cavity 1270 in fluid communication with the inflation lumen 1235, wherein the balloon cavity 1270 may be defined by the distal balloon leg 1268 and the proximal balloon leg 1267.

In another example, FIG. 13 illustrates, by means of a longitudinal cross-sectional view, an example of an elongate dilation catheter 1300 with an elongate flexible tip 1305, wherein a proximal tip end 1372 may be adjacently attached to a distal body end 1325 via a distal face 1307. The dilation catheter 1300 may be composed of an elongate body 1320. The elongate body 1320 may have a longitudinal axis extending between a proximal body end 1322, and the distal body end 1325. In addition, the elongate body 1320 may define parallel dual lumens, a wire guide lumen 1330 and an inflation lumen 1335, wherein the lumens 1330 and 1335 extend longitudinally through the elongate body 1320. The wire guide lumen 1330 extends through the distal body end 1325, whereas the inflation lumen 1335 extends to an intermediate region 1323. The intermediate region 1323 may be positioned between the proximal body end 1322 and the distal body end 1325.

The dilation catheter 1300 also may be fitted with a balloon 1345 having a proximal balloon leg 1350 and a distal balloon leg 1355. The proximal balloon leg 1350 may be attached to the intermediate region 1323. The distal balloon leg 1355 may be adjacently attached to the distal body end 1325, forming a distal bonding region 1360, wherein the distal bonding region 1360 may form the distal face 1307. In one configuration, the distal face 1307 may comprise an angular groove, circumscribing the wire guide lumen 1330. In another configuration, the distal face 1307 comprises a flat surface, circumscribing the wire guide lumen 1330 (not shown). The balloon 1345 may have a balloon cavity 1370 in fluid communication with the inflation lumen 1335, wherein the balloon cavity 1370 is defined by the distal balloon leg 1355 and the proximal balloon leg 1350.

The elongate flexible tip 1305 may comprise a longitudinal axis extending between the proximal tip end 1372 and a distal tip end 1373. A corrugated region 1375 may be located between the proximal tip end 1372 and the distal tip end 1373. The distal tip end 1373 may be integral with a rounded distal end 1380 or the rounded distal end 1380 may be a separate piece or component that is attached to the distal tip end 1373. The proximal tip end 1372 may be adjacently attached to the distal body end 1325 via the distal face 1307. Additionally, the elongate flexible tip 1305 may comprise a tip lumen 1377 extending from the proximal tip end 1372 through the rounded distal end 1380, wherein the tip lumen 1377 is aligned with the wire guide lumen 1330.

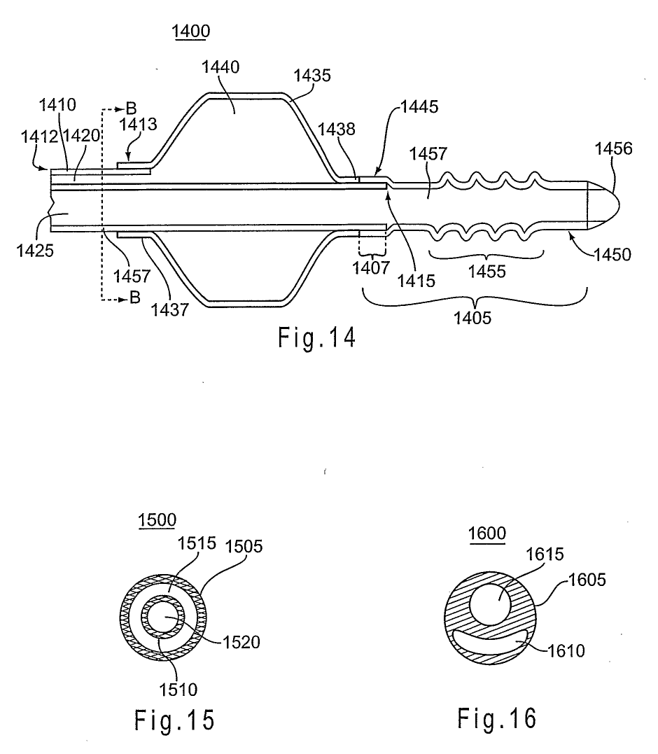

In another example, FIG. 14 illustrates, by means of a longitudinal cross-sectional view, an example of an elongate dilation catheter 1400 with an elongate flexible tip 1405, wherein a proximal tip end 1445 may be adjacently attached to a distal body end 1415 via an external mounting shoulder 1407. The dilation catheter 1400 may be composed of an elongate body 1410. The elongate body 1410 may have a longitudinal axis extending between a proximal body end 1412, and the distal body end 1415. In addition, the elongate body 1410 may define parallel dual lumens, an inflation lumen 1420 and a wire guide lumen 1425, wherein the lumens 1420 and 1425 extend longitudinally through the elongate body 1410. The wire guide lumen 1425 extends through the distal body end 1415, whereas the inflation lumen 1420 extends to an intermediate region 1413. The intermediate region 1413 may be positioned between the proximal body end 1412 and the distal body end 1415.

The dilation catheter 1400 may be fitted with a balloon 1435, having a proximal balloon leg 1437 and a distal balloon leg 1438. The proximal balloon leg 1437 may be attached to the intermediate region 1413. The distal balloon leg 1438 may be adjacently attached to the distal body end 1415, forming the external mounting shoulder 1407. The balloon 1435 may have a balloon cavity 1440 in fluid communication with the inflation lumen 1420, wherein the balloon cavity 1440 is defined by the proximal balloon leg 1437 and the distal balloon leg 1438.

The elongate flexible tip 1405 may comprise a longitudinal axis extending between the proximal tip end 1445 and a distal tip end 1450. A corrugated region 1455 may be located between the proximal tip end 1445 and the distal tip end 1450. The proximal tip end 1445 may be adjacently attached to the distal body end 1415 and the distal balloon leg via the external mounting shoulder 1407. Furthermore, the distal tip end 1450 may be integral with a rounded distal end 1456 or the rounded distal end 1456 may be a separate piece or component that is attached to the distal tip end 1450. Additionally, the elongate flexible tip 1405 may comprise a tip lumen 1457 extending from the proximal tip end 1445 through the rounded distal end 1456, wherein the tip lumen 1457 is aligned with the wire guide lumen 1425.

FIG. 15 illustrates a cross-sectional view through lines A-A of FIGS. 3 thru 9. The dilation catheters of FIGS. 3 thru 9 may comprise an elongate outer body 1505 and an elongate inner body 1510. The outer body 1505 and the inner body 1510 may define an outer lumen 1515 therebetween. The inner body 1510 may contain a single inner lumen 1520.

FIG. 16 illustrates a cross-sectional view through lines B-B of FIGS. 10 thru 14. The dilation catheters of FIGS. 10 thru 14 may comprise an elongate outer body 1605, wherein the elongate body 1605 may contain parallel dual lumens, an inflation lumen 1610 and a wire guide lumen 1615.

It is therefore intended that the foregoing detailed description be regarded as illustrative rather than limiting, and that it be understood that it is the following claims, including all equivalents, that are intended to define the spirit and scope of this invention.

1. An elongate flexible catheter tip comprising:
a longitudinal axis extending between a proximal tip end and a distal tip end; and
a corrugated region located between the proximal tip end and the distal tip end.

2. The elongate flexible tip of claim 1, wherein the elongate flexible tip comprises a tube member that defines a lumen.

3. The elongate flexible tip of claim 1, further comprising a lumen extending longitudinally throughout the elongate flexible tip body.

4. The elongate flexible tip of claim 1, further comprising a rounded distal end.

5. An elongate flexible catheter tip according to claims 1, 2, 3 or 4, wherein the proximal tip end is adjacently attached to an inner distal end of a dilation catheter, the dilation catheter comprising:
an elongate outer body comprising a longitudinal axis extending between an outer proximal end and an outer distal end;
an elongate inner body, comprising:
a proximal region located within the outer body and extending between the outer proximal end and the outer distal end,
a distal region extending past the outer distal end and comprising the inner distal end, and
an inner lumen contained within the inner body;
an outer lumen defined by the outer body and the inner body; and
a balloon comprising:
a proximal balloon leg attached to the outer distal end;
a distal balloon leg attached to a distal end of the dilation catheter; and
a balloon cavity defined by the proximal balloon leg and the distal balloon leg and in fluid communication with the outer lumen.

6. The elongate flexible catheter tip of claim 5, wherein:
the proximal tip end is attached to the inner distal end at a tip-end attachment;
the distal balloon leg is attached across the tip-end attachment; and
the distal tip end comprises a rounded distal end.

7. The elongate flexible catheter tip of claim 5, wherein:
the proximal tip end is attached to the inner distal end at a tip-end attachment;
the distal balloon leg is attached to the distal region, proximal to the tip-end attachment; and
the distal tip end comprises a rounded distal end.

8. The elongate flexible catheter tip of claim 5, wherein:
the elongate inner body comprises a braided coil reinforcing the elongate inner body;
the inner distal end is integral with the elongate flexible tip;
the distal balloon leg is attached to the distal region, proximal to the corrugated region; and
the distal tip end comprises a rounded distal end.

9. The elongate flexible catheter tip claim 8, wherein:
the braided coil is of uniform tightness, throughout the elongate inner body.

10. The elongate flexible catheter tip of claim 8, wherein:
the braided coil is of varying tightness throughout the elongate inner body.

11. The elongate flexible catheter tip of claim 5, wherein:
the distal balloon leg is attached to the inner distal end, providing a distal bonding region, wherein the distal bonding region has a distal face circumscribing the inner distal end;
the proximal tip end is attached to the distal face; and
the distal tip end comprises a rounded distal end.

12. The elongate flexible catheter tip of claim 5, wherein:
the distal balloon leg is attached to the distal region;
the elongate inner body comprises an inner material and an outer material, wherein:
the outer material has a lower durometer than the inner material;
the outer material extends distally beyond the inner material and is integral with the tip;
the distal tip end comprises a rounded distal end.

13. The elongate flexible catheter tip of claim 5, wherein:
the distal balloon leg is attached to the distal region, such that the distal region extends beyond the balloon leg, comprising an external mounting shoulder;
the flexible tip is attached to the external mounting shoulder; and
the distal tip end comprises a rounded distal end.

14. The elongate flexible catheter tip of claim 5, wherein:
the distal balloon leg is attached adjacent to the inner distal end and the proximal end of the flexible tip.

15. The elongate flexible catheter tip of claim 1, wherein a tip lumen is defined by the elongate flexible catheter tip, the tip lumen being aligned with a wire guide lumen and the proximal tip end is adjacently attached to a distal body end of a dilation catheter, the dilation catheter comprising:
a longitudinal axis extending between a proximal body end and the distal body end;
an inflation lumen and the wire guide lumen, wherein the inflation lumen and the wire guide lumen are parallel and are defined by the elongate body;
an intermediate region positioned between the proximal body end and the distal body end; and
a balloon comprising:
a proximal balloon leg attached to the intermediate region;
a distal balloon leg adjacently attached to the distal body end; and
a balloon cavity defined by the proximal balloon leg and the distal balloon leg, and in fluid communication with the inflation lumen.

16. An elongate flexible catheter tip according to claims 1, 2, 3 or 4, wherein the elongate flexible tip comprises a material selected from the group consisting of nylon, polyether-block co-polyamide polymers, polyethylene, polyvinyl chloride, polystyrene, silicon co-polymer, polyolefin, polyurethane and combinations thereof.

17. The elongate flexible catheter tip of claim 15, wherein:
the proximal tip end is adjacently attached to the distal body end forming a tip-end attachment;
the distal balloon leg is adjacently attached to the distal body end, the proximal tip end, and the tip-end attachment; and
the distal tip end is integral with a rounded distal end.

18. The elongate flexible catheter tip of claim 15, wherein:
the proximal tip end is adjacently attached to the distal body end forming a tip-end attachment;
the distal balloon leg is adjacently attached to the distal body end; and
the distal tip end is integral with a rounded distal end.

19. The elongate flexible catheter tip of claim 15, wherein:
the distal balloon leg is adjacently attached to the distal body end, forming a distal bonding region, wherein the distal bonding region has a distal face that circumscribes the wire guide lumen;
the proximal tip end is adjacently attached to the distal body end and the distal balloon leg via the distal face; and
the distal tip end is integral with a rounded distal end.

20. The elongate flexible catheter tip of claim 15, wherein:
the distal balloon leg is adjacently attached to the distal body end, forming an external mounting shoulder;
the proximal tip end is adjacently attached to distal body end and the distal balloon leg via the external mounting shoulder; and
the distal tip end comprises a rounded distal end.

21. The elongate flexible catheter tip of claim 15, wherein:
the distal balloon leg is adjacently attached to the distal body end and the proximal tip end.

22. An elongate flexible catheter tip as in claims 1, 2, 3 or 4, wherein the corrugated region comprises an accordion corrugation.

23. The elongate flexible catheter tip of claim 22, wherein the corrugated region further comprises a plurality of ridges interspersed with a plurality of grooves.

24. The elongate flexible catheter tip of claim 22, wherein the corrugated region further comprises a plurality of grooves.

25. The elongate flexible catheter tip of claim 22, wherein the corrugated region further comprises a plurality of ridges.

26. The elongate flexible catheter tip of claim 23, wherein the wall thickness is substantially the same throughout the tube member, including the corrugated region.

27. The elongate flexible catheter tip of claim 26, wherein the ridges have an outer diameter that is greater than an outer diameter of the tube member and the grooves have an inner diameter that is smaller than an inner diameter of the tube member.

28. The elongate flexible catheter tip of claim 26, wherein the ridges have an outer diameter that is greater than an outer diameter of the tube member and the grooves have an inner diameter that is substantially the same as an inner diameter of the tube member.

29. An elongate flexible catheter tip as in claims 1, 2, 3 or 4, wherein the corrugated region comprises a helical corrugation.
